(19) United States
(12) Patent Application Publication
(10) Pub. No.: US 2023/0240729 A1
(43) Pub. Date: Aug. 3, 2023

(54) ARTHRODESIS NAIL

(71) Applicant: TST RAKOR VE TIBBi ALETLER SANAYi VE TICARET LIMITED SIRKETI, Pendik - Istanbul (KR)

(72) Inventors: Engin Eceviz, Kadikoy Istanbul (TR); Oonder Ismet Kilicoglu, Uskudar Istanbul (TR)

(21) Appl. No.: 17/925,552

(22) PCT Filed: Oct. 19, 2021
(86) PCT No.: PCT/TR2021/051065

§ 371 Date: Nov. 15, 2022

(30) Foreign Application Priority Data
Oct. 30, 2020 (TR) 2020/17292

(51) Int. Cl. A61B 17/72

(52) U.S. Cl. CPC A61B 17/7283; A61B 17/7291; A61B 17/7225; A61B 17/7241

(57) ABSTRACT

The invention relates to nails used for ankle deformities and joint disorders characterized in that it comprises, in order to form an ankle arthrodesis nail (1) acting as a fixing nail passing through inside the canals of calcaneus, talus and tibia bones (19, 18, 17), providing a more rapid recovery and reducing the infection risk, at least three and at least two screw holes in lockable structure in its distal part and in its proximal part, respectively, arthrodesis nail (1) providing the closure of the inter-bone gap and an easier fusion by means of the compression effect during being fastened upon the bones, protrusions (15) extending from its proximal to its distal, and slit (12) or slits (12) at the proximal tip of the arthrodesis nail (1) to decrease the stress cracks that might occur due to the tip of the arthrodesis nail (1).

### TECHNICAL FIELD

The present invention relates to arthrodesis nails used for ankle deformities and joint disorders.

In particular, the invention is ankle arthrodesis nail that enables overcoming various technical problems, designed for ankle deformities and joint disorders and working as a fixing nail passing intramedullary through calcaneus, talus and tibia bones.

### DESCRIPTION OF STATE OF THE ART

Arthrodesis, ensuring the joint ossification between two bones artificially by means of surgery. Ankle arthrodesis is an established therapeutic option in the treatment of certain pathological conditions of the ankle.

Although indications expand along years, basic principles remained unchanged; adjusting the deformity, relieving the pain, preventing amputation and ensuring stability.

For ankle arthrodesis, methods like external fixator, plate-screwing, intramedullary nail are used.

There are post plate-screwing risks such as late motion formation, excess incision and thereby infection.

External fixators have negative sides such as remaining outside of the skin, preventing the movements and therefore the comfort of the patient.

Due to the complications and rigidity insufficiency emerging as a result of other methods, nail use increases day by day. New technological developments removing these negativities are needed.

One feet problem of sareo and diabetics patients is the low bone intensity and quality. The present arthrodesis nails applied to these types of patients cannot provide sufficient stability due to low bone quality and prevent formation of fusion (bone union).

As present nails (Retrodesis Nail) have a cylindrical structure, calcaneus and talus bones may rotate on the nail. This movement in turn prevents fusion (bone union). Therefore, joint ossification cannot happen. New technical developments removing these negative conditions are needed.

The patent application numbered TR 2009/06698 discloses, for the arthrodesis reconstruction of the calcaneus structures, a lock nail system in the form of a cylindrical geometric part with a tapering end, the stem of the said structure being connected to a claw adapted to be fit on a guide pin placed in talus, said guide pin forming a reference axis crossing a plane defined by the axis of the guide hole and the axis of one of the holes in the tampon, said lock nail system acting as a support for the location of the nail and calcaneus-talar screws of the said guide pin to be set by means of a handle on the tip of the stem. However, a different technical structure is mentioned. There is a need for a different nail structure that resolves the above-described problems.

### DESCRIPTION OF THE AIMS OF THE INVENTION

By looking at the state of the art, the aim of the invention is to develop an ankle arthrodesis nail that resolves the negativities present in the current structures.

Another aim of the invention is to develop the arthrodesis nail that ensures to resolve the ankle problems encountered in connection with the sareo foot and diabetics diseases.

Another aim of the invention is to remove the problems of the current arthrodesis nails, namely not being able to provide the sufficient stability and fusion (bone union) due to low bone quality of the patient.

Another aim of the invention is to develop the arthrodesis nail used in clearing up the ankle deformities and joint disorders.

Another aim of the invention is to develop a new arthrodesis nail ensuring the patient enters into a more rapid recovery process for adjusting the deformity, relieving the pain, preventing the amputation and providing stability.

Another aim of the invention is to develop an ankle arthrodesis nail acting as a fixing nail passing through inside the canals of calcaneus, talus and tibia, providing a more rapid recovery and decreasing the infection risk.

In order to achieve said aims, a new arthrodesis nail bringing solutions to the current problems and contributing to the recovery process of the patient.

### DESCRIPTION OF THE FIGURES

FIG. 1 is the drawing of the medial-lateral view of the arthrodesis nail placed inside the bone in a representative embodiment of the invention.

FIG. 2 is the drawing of the posterior-anterior view of the arthrodesis nail placed inside the bone in a representative embodiment of the invention.

FIG. 3 is the sectional A-A drawing of the posterior-anterior view of the arthrodesis nail in a representative embodiment of the invention.

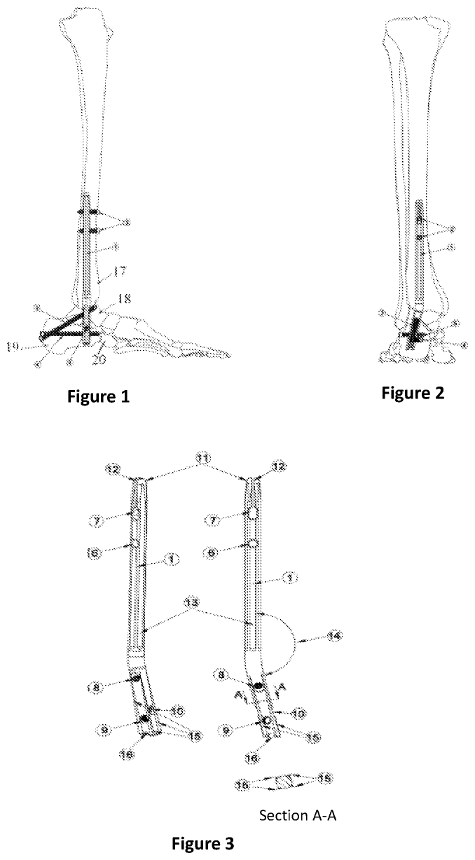

### REFERENCE NUMERALS

| | |
|---|---|
| 1 | Arthrodesis nail |
| 2 | Anterior-posterior oriented nail fixing |
| | screw |
| 3 | Nail fixing screw directed from |
| | calcaneus to talus |
| 4 | Nail fixing screw directed from |
| | calcaneus to cuboid |
| 5 | Nail fixing screw directed from lateral |
| | to medial |
| 6 | Anterior-posterior oriented nail fixing |
| | screw hole |
| 7 | Dynamic anterior-posterior oriented |
| | nail fixing screw dynamic hole |
| 8 | Nail fixing screw locking hole directed |
| | from calcaneus to talus |
| 9 | Nail fixing screw locking hole directed |
| | from calcaneus to cuboid |
| 10 | Nail fixing screw locking hole directed |
| | from lateral to medial |
| 11 | Rounded tip |
| 12 | Slit |
| 13 | Groove |
| 14 | 12° angle |
| 15 | Protrusion |
| 16 | Nail guide connection slots |
| 17 | Tibia bone |
| 18 | Talus bone |
| 19 | Calcaneus bone |
| 20 | Cuboid bone |

### DETAILED DESCRIPTION OF THE INVENTION

Developed arthrodesis nail (1) includes 3 screw holes in lockable structure in its distal part. There are nail fixing screw locking hole directed from calcaneus to talus (8), nail fixing screw locking hole directed from calcaneus to cuboid (9) and nail fixing screw locking hole directed from lateral to medial (10).

Anterior-posterior oriented nail fixing screw (2), nail fixing screw directed from calcaneus to talus (3), nail fixing screw directed from calcaneus to cuboid (4) and nail fixing screw directed from lateral to medial (5) are used.

Through the locked nail fixing screw locking hole directed from calcaneus to talus (8), the nail fixing screw directed from calcaneus to talus (3) can be sent from calcaneus bone (19) to talus bone (18). Through the locked nail fixing screw locking hole directed from calcaneus to cuboid (9), the nail fixing screw directed from calcaneus to cuboid (4) can be sent from calcaneus bone (19) to cuboid bone (20).

Nail fixing screw directed from lateral to medial (5) can be sent from the lateral of the calcaneus bone (19) to its medial from the nail fixing screw locking hole directed from lateral towards medial (10).

In the distal part of the developed arthrodesis nail (1), there are 4 spired, sharp protrusions (15) preventing the bone rotation. This spired, sharp protrusions (15) are fastened upon calcaneus and talus bones (19, 18) and prevent the bones from rotating on the nail. This is amongst the most important features of the arthrodesis nail (1) and is unique.

These protrusions (15) are also in a conic structure extending from the proximal to the distal of the arthrodesis nail (1). This conic structure provides a compression effect during the fastening of the arthrodesis nail (1) upon the bone. With this compression effect, the gap between the bones is closed and fusion occurs more easily. This is amongst the most important features of the arthrodesis nail (1) and is unique.

There is a 12° angle (14) appropriate for the anato mic positions of the calcaneus and tibia bones (19,17). There are straight or angular nail designs. Taking calcaneus entry point into account, it might be technically difficult to obtain a 5 degree valgus in the ankle with straight nails. For this reason, nail designs with inclinations in the entry point are of preference for both ensuring the appropriate array and obtaining a more stable fixing in calcaneus.

Arthrodesis nail (1) ensures the ankle joint fixation by passing through the medulla canals of calcaneus, talus and tibia bones (19, 18, 17).

In the proximal of the arthrodesis nail (1), there are 6 number of grooves (13). These grooves prevent the rotation the tibia bone (17) may make on the arthrodesis nail (1).

In the proximal of the arthrodesis nail (1), there are two screw holes; static anterior-posterior oriented nail fixing screw hole (6) and dynamic anterior-posterior oriented nail fixing screw hole (7), directed from the anterior to the posterior of the tibia bone (17).

Nail guide connection slots (16) are formed on the lower part of the arthrodesis nail (1).

The proximal tip of the arthrodesis nail (1) is rounded, and the rounded tip (11) was formed so as to ensure that the arthrodesis nail (1) move in the medulla canal easily.

There is a slit (12) on the proximal tip of the arthrodesis nail (1). This slit (12) is designed to reduce the stress cracks that may occur due to the arthrodesis nail (1) tip. This is one of the most important features of the arthrodesis nail (1). It is a feature that is not present in other applications.

Arthrodesis nails (1) in 6 different diameters and sizes has been designed in line with the patient bone sizes. They can be formed in different sizes as well.

The invention relates to nails used for ankle deformities and joint disorders characterized in that it comprises, in order to form an ankle arthrodesis nail (1) acting as a fixing nail passing through inside the canals of calcaneus, talus and tibia bones (19, 18, 17), providing a more rapid recovery and reducing the infection risk, at least three and at least two screw holes in lockable structure in its distal part and in its proximal part, respectively, arthrodesis nail (1) providing the closure of the inter-bone gap and an easier fusion by means of the compression effect during being fastened upon the bones, protrusions (15) extending from its proximal to its distal, and slit (12) or slits (12) at the proximal tip of the arthrodesis nail (1) to decrease the stress cracks that might occur due to the tip of the arthrodesis nail (1).

It comprises a 12° angle (14) appropriate for the anatomic positions of the calcaneus and tibia bones (19,17). In its distal part, it comprises a nail fixing screw locking hole directed from calcaneus to talus (8), nail fixing screw locking hole directed from calcaneus to cuboid (9) and nail fixing screw locking hole directed from lateral to medial (10).

In its distal and proximal parts, it comprises anterior-posterior oriented nail fixing screw (2), nail fixing screw directed from calcaneus to talus (3), nail fixing screw directed from calcaneus to cuboid (4) and nail fixing screw directed from lateral to medial (5).

It comprises the nail fixing screw directed from calcaneus to talus (3) going from calcaneus bone (19) to talus bone (18) through the locked nail fixing screw locking hole directed from calcaneus to talus (8).

It comprises the nail fixing screw directed from calcaneus to cuboid (4) going from calcaneus bone (19) to cuboid bone (20) through the locked nail fixing screw locking hole directed from calcaneus to cuboid (9).

It comprises nail fixing screw directed from lateral to medial (5) that is sent from the lateral of the calcaneus bone (19) to its medial from the nail fixing screw locking hole directed from lateral towards medial (10).

It comprises spired, sharp protrusions (15) in conic structure that are fastened upon calcaneus and talus bones (19, 18) to prevent the bone rotations on the nail and extending from proximal to distal preventing the bone rotation in the distal part. It comprises grooves (13) in the proximal of arthrodesis nail (1) preventing the rotation of the tibia bona (17) on the arthrodesis nail (1).

In the proximal of the arthrodesis nail (1), it comprises the static anterior-posterior oriented nail fixing screw hole (6) and dynamic anterior-posterior oriented nail fixing screw hole (7), directed from the anterior to the posterior of the tibia bone (17).

In the proximal tip of the arthrodesis nail (1), it comprises the rounded tip (11) formed so as to ensure that arthrodesis nail (1) move in the medulla canal easily. It comprises nail guide connection slots (16) in the lower part of the arthrodesis nail (1) and slit (12) in the proximal tip of the arthrodesis nail (1) to reduce the stress cracks due to the tip of the arthrodesis nail (1).

1. Nails used for ankle deformities and joint disorders characterized in that it comprises, in order to form an ankle arthrodesis nail (1) acting as a fixing nail passing through inside the canals of calcaneus, talus and tibia bones (19, 18, 17), providing a more rapid recovery and reducing the infection risk,
at least three and at least two screw holes in lockable structure in its distal part and in its proximal part, respectively,
arthrodesis nail (1) providing the closure of the inter-bone gap and an easier fusion by means of the compression effect during being fastened upon the bones, protrusions (15) extending from its proximal to its distal,
and slit (12) or slits (12) at the proximal tip of the arthrodesis nail (1) to decrease the stress cracks that might occur due to the tip of the arthrodesis nail (1).

2. Arthrodesis nail (1) according to claim 1, wherein; it comprises
a 12° angle (14) appropriate for the anatomic posit ions of the calcaneus and tibia bones (19,17).

3. Arthrodesis nail (1) according to claim 1, wherein; it comprises
in its distal part, a nail fixing screw locking hole directed from calcaneus to talus (8), nail fixing screw locking hole directed from calcaneus to cuboid (9) and nail fixing screw locking hole directed from lateral to medial (10).

4. Arthrodesis nail (1) according to claim 1, wherein; it comprises
in its distal and proximal parts, anterior-posterior oriented nail fixing screw (2), nail fixing screw directed from calcaneus to talus (3), nail fixing screw directed from calcaneus to cuboid (4) and nail fixing screw directed from lateral to medial (5).

5. Arthrodesis nail (1) according to claim 1, wherein; it comprises
the nail fixing screw directed from calcaneus to talus (3) going from calcaneus bone (19) to talus bone (18) through the locked nail fixing screw locking hole directed from calcaneus to talus (8).

6. Arthrodesis nail (1) according to claim 1, wherein; it comprises
the nail fixing screw directed from calcaneus to cuboid (4) going from calcaneus bone (19) to cuboid bone (20) through the locked nail fixing screw locking hole directed from calcaneus to cuboid (9).

7. Arthrodesis nail (1) according to claim 1, wherein; it comprises
nail fixing screw directed from lateral to medial (5) that is sent from the lateral of the calcaneus bone (19) to its medial from the nail fixing screw locking hole directed from lateral towards medial (10).

8. Arthrodesis nail (1) according to claim 1, wherein; it comprises
spired, sharp protrusions (15) in conic structure that are fastened upon calcaneus and talus bones (19, 18) to prevent the bone rotations on the nail and extending from proximal to distal preventing the bone rotation in the distal part.

9. Arthrodesis nail (1) according to claim 1, wherein; it comprises
grooves (13) in the proximal of arthrodesis nail (1) preventing the rotation of the tibia bona (17) on the arthrodesis nail (1).

10. Arthrodesis nail (1) according to claim 1, wherein; it comprises
in the proximal of the arthrodesis nail (1), the static anterior-posterior oriented nail fixing screw hole (6) and dynamic anterior-posterior oriented nail fixing screw hole (7), directed from the anterior to the posterior of the tibia bone (17).

11. Arthrodesis nail (1) according to claim 1, wherein; it comprises
In the proximal tip of the arthrodesis nail (1), the rounded tip (11) formed so as to ensure that arthrodesis nail (1) move in the medulla canal easily.

12. Arthrodesis nail (1) according to claim 1, wherein; it comprises
nail guide connection slots (16) in the lower part of the arthrodesis nail (1).

13. Arthrodesis nail (1) according to claim 1, wherein; it comprises
slit (12) in the proximal tip of the arthrodesis nail (1) to reduce the stress cracks due to the tip of the arthrodesis nail (1).
